SEQUENCE LISTING (6 sequences)

SEQ ID NO: 1 - DNA, 20 bp, Artificial Sequence
  Synthetic DWV Sense Primer
cgaaaccaacttctgaggaa

SEQ ID NO: 2 - DNA, 20 bp, Artificial Sequence
  Synthetic DWV Antisense Primer
tcgacaattttcggacatca

SEQ ID NO: 3 - DNA, 20 bp, Artificial Sequence
  Synthetic Dorsal Forward Primer
tcggatggtgctacgagcga

SEQ ID NO: 4 - DNA, 23 bp, Artificial Sequence
  Synthetic Dorsal Reverse Primer
agcatgcttctcagcttctgcct

SEQ ID NO: 5 - DNA, 23 bp, Artificial Sequence
  Synthetic Apidaecin Forward Primer
ttttgccttagcaattcttgttg

SEQ ID NO: 6 - DNA, 21 bp, Artificial Sequence
  Synthetic Apidaecin Reverse Primer
gaaggtcgagtaggcggatct

(19) United States
(12) Patent Application Publication
(10) Pub. No.: US 2021/0199658 A1
(43) Pub. Date: Jul. 1, 2021

(54) DETECTION OF LIPASE ACTIVITY IN HONEY BEES

(71) Applicant: The United States of America, as Represented by the Secretary of Agriculture, Washington, DC (US)

(72) Inventors: Yanping Chen, Laurel, MD (US); Matthew C. Heerman, Lanham, MD (US); Steven C. Cook, Capital Heights, MD (US); Jay D. Evans, Harwood, MD (US)

(21) Appl. No.: 17/135,415

(22) Filed: Dec. 28, 2020

Related U.S. Application Data
(60) Provisional application No. 62/955,156, filed on Dec. 30, 2019.

(51) Int. Cl. G01N 33/573

(52) U.S. Cl. CPC G01N 33/573; G01N 33/6839; G01N 2333/43565

(57) ABSTRACT

This disclosure relates generally to novel kits for measuring insect health, and to methods of making and using such compositions. More specifically, the invention relates to novel kits for a rapid and high-throughput measurement of lipase activity levels in insects, and the correlation of the measured lipase activity levels with insect stress.

### CROSS-REFERENCE TO RELATED APPLICATIONS

This application claims priority to U.S. Provisional Patent Application No. 62/955,156, filed Dec. 30, 2019. The content of this provisional patent application is hereby expressly incorporated by reference in its entirety.

### FIELD OF THE INVENTION

This disclosure relates generally to novel kits and methods for measuring insect lipase activity in insects. The disclosure also relates to the correlation of the measured lipase activity with insect stress.

### SEQUENCE LISTING

The instant application contains a Sequence Listing which has been submitted via EFS-Web as ASCII compliant text file format (.txt), and is hereby incorporated by reference in its entirety. The ASCII file was created on Dec. 30, 2019, is named Sequence Listing, and has 2 kilobytes. This Sequence Listing serves as paper copy of the Sequence Listing required by 37 C.F.R. § 1.821(c) and the Sequence Listing in computer-readable form (CRF) required by 37 C.F.R. § 1.821(e). A statement under 37 C.F.R. § 1.821(f) is not necessary.

### BACKGROUND OF THE INVENTION

Pollination of food crops and wildlife habitats is an indispensable part of the global agricultural economy and ecology. The European honey bee, Apis mellifera, provides pollination to approximately one-third of human food crops. Globally, these pollination services are estimated to exceed US$153 billion every year.

Environmental changes, pesticides, pollutants, parasites, diseases, decreased genetic diversity, and malnutrition have all been linked to bee population declines in the industrialized world. It is thought that the major driver of pollinator decline is loss and degradation of flower-rich habitats, and their replacement with extensive monocultures. The increased exposure of bees to anthropogenic-induced stressors such as the movement and trade of pollinators across the globe, increased pesticide usage, and environmental changes are other stressors that detract from the overall health and survivorship of honey bee colonies.

Bees are host to a diversity of viral species and strains. Deformed wing virus (DWV) is closely associated with characteristic wing deformities, abdominal bloating, paralysis, and rapid mortality of emerging adult bees. The virus is a worldwide bee disease often associated with high Varroa mite populations. In the absence of Varroa, DWV normally persists at low levels within the bee colony with no detrimental effect. The DWV is a member of the Iflaviridae family, and can be found in all bee life stages from egg to adult, and in the glandular secretions used to feed larvae and the queen. Isolated from adult deformed bees in 1982 in Japan, DWV was given its name after the symptoms with which it was closely associated.

Besides DWV, other viruses and viral families have been identified in populations of bees and their parasites. For example, Israeli acute paralysis virus (IAPV), Kashmir bee virus (KBV), acute bee paralysis virus (ABPV), black queen cell virus (BQCV), DWV, Kakugo virus, Varroa destructor virus-1/DWV-B, sacbrood virus (SBV), slow bee paralysis virus, Lake Sinai virus (LSV), Tobacco ringspot virus (TRSV), Ganda bee virus, Apis mellifera filamentous virus, Osmida cornuta nudivirus (OcNV), Bee macula-like virus, chronic bee paralysis virus (CBPV), and Scaldis River Bee Virus (SRBV), among others.

Recent studies have demonstrated that the tripartite interaction between honey bees, Varroa destructor mites, and the viruses vectored by the mites contribute heavily to the hive losses observed. Because the mite and virus are symbiotically linked, rapid, and early detection of viruses can inform beekeepers on what interventions to make to rescue their hives from collapse. The currently used method for detecting DWV in honey bees was developed and published by the USDA-ARS-BRL in 2004 (Chen, Y. P, et al., 2004, “Quantitative Analysis of Deformed Wing Virus Infection in the Honey Bee, Apis mellifera L. by real-time RT-PCR,” Appl. Environ. Microbiol. 71: 436-441). While this method is very accurate at diagnosing and quantifying DWV infection and titers, it suffers from being relatively expensive and time-consuming when compared to fluorescent resonance energy transfer (FRET)-based assays.

In 2019 at least two groups offer assays to test for the presence of pathogens in honey bees the United States. The North Carolina State Extension Service at the North Carolina State University offers an Apiary Pathogen Screen of up to 10 pooled colonies for US$220.00. This screen identifies the presence and relative levels of ABPV; BQCV; DWV genotypes A and B (DWV(A&B)); IAPV; LSV; Trypanosomes; and two Nosema species. The Bee Informed Partnership in association with the University of Maryland offers diagnostic test kits where 16 hives are sampled, 8 weak or crashing colonies and 8 healthy colonies, for US$475.00 plus shipping for diagnostic testing, and US$525.00 plus shipping for expedited results. Live bees are tested for viral loads from KBV; ABPV; IAPV; DWV; LSV-2; CBPV; BQCV; Nosema; and Varroa. Testing pollen, wax, or bees for 170 known pesticides is offered for an additional US$780.00.

To date there are no quick and economic methods for diagnosing and quantifying bee lipase activity. Thus, a rapid and relatively inexpensive method for diagnosing and quantifying bee colony lipase activity is needed.

### SUMMARY OF THE INVENTION

The inventors have devised a novel method for measuring lipase activity levels in insects. The inventors surprisingly found that the lipase activity levels measured using this novel method correlate with in the insects' stress levels. Thus, the inventors have devised a method for determining and quantifying insect stress.

In an embodiment, the invention relates to a kit comprising a fluorogenic triglyceride comprising a dye and a quencher, a substrate reaction buffer comprising Zwittergent detergent in Phosphate Buffered Saline (PBS), and optionally a sample buffer comprising bovine serum albumin (BSA) and Zwittergent detergent in PBS.

In some embodiments of the invention, the dye in the kit is a boron-dipyrromethene (BODIPY) dye; an ALEXA FLUOR dye; a PACIFIC GREEN dye; an OREGON GREEN dye; Fluorescein; fluorescein isocyanate; tetrachlorfluorescein; a CAL fluor; 4,5-dichloro-dimethoxy-fluorescein; hexachloro-fluorescein; Evans Blue; or DYLIGHT fluorescent dye. In some embodiments of the invention, the dye is a BODIPY dye.

In some embodiments of the invention, the quencher in the kit is 4-((4-dimethylamino) phenyl)azo)benzoic acid (DABCYL acid); 4′-(2-Nitro-4-toluyldiazo)-2′-methoxy-5′-methyl-azobenzene-4″-(N-ethyl)-N-ethyl-2-cyanoethyl-(N,N-diisopropyl)-phosphoramidite; or a BLACK HOLE quencher. In some embodiments of the invention, the quencher is DABCYL acid. In some embodiments of the invention, the dye is a BODIPY dye and the quencher is a DABCYL acid quencher.

In some embodiments of the invention, the substrate reaction buffer in the kit comprises about 0.005% Zwittergent detergent. In some embodiments of the invention, the sample buffer in the kit comprises about 0.0015% BSA, and about 0.06% Zwittergent detergent in 4×PBS. In some embodiments of the invention, each of the sample buffer, the substrate reaction buffer, and the fluorogenic triglyceride comprising a dye and a quencher in the kit are in separate containers.

In an embodiment, the invention relates to a method for determining lipase activity levels in an insect biological sample. The method comprises mixing an insect biological sample with a sample buffer comprising BSA and Zwittergent detergent in PBS to obtain a biological sample solution; mixing a fluorogenic triglyceride comprising a dye and a quencher with a substrate reaction buffer comprising Zwittergent detergent in PBS to form a working solution; adding the biological sample solution to a well of a well plate; adding the working solution to the biological sample solution in the well of the well plate to form a mixture; incubating the well plate containing the mixture; and measuring the emitted fluorescence; where the measured emitted fluorescence is an indication of the lipase activity in the insect biological sample.

In some embodiments of the invention, in the method for determining lipase activity levels in an insect biological sample, the dye in the fluorogenic triglyceride is a BODIPY dye; an ALEXA FLUOR dye; a PACIFIC GREEN dye; an OREGON GREEN dye; Fluorescein; fluorescein isocyanate; tetrachlorfluorescein; a CAL fluor; a DYLIGHT fluor; 4,5-dichloro-dimethoxy-fluorescein; hexachloro-fluorescein; or Evans Blue. In some embodiments of the invention, in the method for determining the lipase activity levels in an insect biological sample, the quencher in the fluorogenic triglyceride is 4-((4-dimethylamino) phenyl)azo)benzoic acid (DABCYL acid); 4′-(2-Nitro-4-toluyldiazo)-2′-methoxy-5′-methyl-azobenzene-4″-(N-ethyl)-N-ethyl-2-cyanoethyl-(N,N-diisopropyl)-phosphoramidite; or a BLACK HOLE quencher. In some embodiments of the invention, in the method for determining lipase activity levels in an insect biological sample, the dye is a BODIPY dye, and the quencher is a DABCYL acid quencher.

In some embodiments of the invention, in the method for determining lipase activity levels in an insect biological sample, the fluorogenic triglyceride comprising a dye and a quencher is present at about 0.31 μM; about 0.62 μM; about 1.24 μM; about 2.48 μM; about 4.96 μM; about 9.92 μM; or about 19.84 μM. In some embodiments of the invention, in the method for determining lipase activity levels in an insect biological sample, the fluorogenic triglyceride comprising a dye and a quencher is present at about 2.48 μM.

In an embodiment, the invention is related to methods of determining lipase activity in Insecta. In some embodiments of the invention, the Insecta is a Coleoptera, a Lepidoptera, a Hymenoptera, or a Diptera. In some embodiments, the invention is related to a method for determining lipase activity in a biological sample from a Hymenoptera. In some embodiments, the invention is related to a method for determining lipase activity in biological sample from a bee. In some embodiments, the invention is related to a method for determining lipase activity in biological sample from a drone bee, a worker bee, or a queen bee. In some embodiments, the invention is related to a method for determining lipase activity in a biological sample from homogenized eggs, homogenized larvae, homogenized pupae, or homogenized adult insects.

In an embodiment, the invention relates to a method for determining lipase activity levels in an insect biological sample. The method comprises mixing an insect biological sample with the sample buffer to obtain a biological sample solution; mixing the fluorogenic triglyceride comprising a dye and a quencher with the substrate reaction buffer to form a working solution; adding the biological sample solution to a well of a well plate; adding the working solution to the biological sample solution in the well of the well plate to form a mixture; incubating the well plate; and measuring the emitted fluorescence; where the measured emitted fluorescence is an indication of the lipase activity in the insect biological sample.

In an embodiment, the invention relates to a method for determining stress in at least one test honey bee. The method comprises measuring the lipase activity levels of at least one test honey bee and of at least one healthy honey bee using the methods of the invention; comparing the measured lipase activity levels of the test honey bee with the measured lipase activity levels of the healthy honey bee; and determining that the test honey bee is under stress if the measured lipase activity levels of the at least one test honey bee are higher than the measured lipase activity levels of the at least one healthy honey bee; or if the slope of the measured lipase activity levels in the test honey bee present a steeper slope than the measured lipase activity levels in the healthy honey bee. In some embodiments of the invention, the stress in the at least one test honey bee is caused by a pathogen; a pesticide; ontogeny; environmental change; or hypoxia. In some embodiments of the invention, the pathogen causing the stress on the test honey bee is DWV, IAPV, KBV, ABPV, BQCV, Kakugo virus, Varroa destructor virus-1/DWV-B, SBV, slow bee paralysis virus, LSV, TRSV, Ganda bee virus, Apis mellifera filamentous virus, OcNV, Bee macula-like virus, CBPV, or SRBV.

### BRIEF DESCRIPTION OF THE DRAWINGS

FIG. 1 depicts a graph of the pupal lipase reaction rate determined using fluorescent lipid substrate for honey bee pupae. Triangles depict results for honey bee pupae injected with PBS solution. Circles depict results for honey bee pupae injected with 10¹⁰ DWV viral particles. X axis depicts the concentration of substrate [S] in μM. Y axis depicts the rate of lipase enzymatic activity obtained from the measured fluorescence. Non-linear Michaelis-Menten was performed to determine statistical significance (P<0.0001; F=33.74, DFn=2, DFd=171). Each data point represents the mean of 12 determinations.

FIG. 2 depicts a graph of the DWV load per bee measured using qPCR, and analyzed using 2^{-ΔΔct} method. Y axis depicts the DWV viral particles per bee. The X axis depicts the sample type, honey bees injected with 10¹⁰ DWV, or honey bees injected with PBS. An unpaired two-way Student's t-test was performed (P<0.0001; t=17.77, DFn=1, DFd=500). The star [★] above the bar denotes the 2^{-ΔΔct} calibrator. Each data point represents the mean of 12 determinations.

FIG. 3 depicts a graph of the linear assay response when using 2.48 μM EnzChek lipase substrate. Squares depict results for honey bees acquired from DWV-infected hives, and circles depict results for honey bees acquired from healthy hives. X axis indicates the time in minutes. Y axis depicts the measured Relative Fluorescent Units (RFU). A one-way ANOVA with a post hoc Tukey test was used to measure significance (P<0.0001; F=17.11, DFn=1, DFd=250). Bars represent standard error. Each data point represents the mean of 12 determinations.

FIG. 4 depicts the total lipid content per bee, measured using the vanillin assay. Y axis depicts the total lipid content per bee in μg/bee. The X axis indicates the sample type, either bees from healthy hives, or bees from DVW-infected hives. Unpaired two-way Student's t-test was performed; (P<0.007; t=2.974, df=22). Bars represent standard error. Each bar represents the mean of 12 determinations.

FIG. 5 depicts a graph of the viral loads in Field Bees measured using qPCR. Y axis depicts the viral load per bee. The X axis depicts the type of bees examined, either from a healthy hive (Healthy), asymptomatic bees from a DWV-infected hive, or deformed nurse bees from a DWV-infected hive. A one-way ANOVA with a post hoc Tukey test was used to measure significance (P<0.0001 between a & b groups; F=4.836, DFn=2, DFd=33). Bars represent standard error. Each data point represents the mean of 12 determinations.

FIG. 6 depicts a graph of the change in Dorsal and Apidaecin gene transcript levels in DWV-injected and PBS-injected bees. Y axis depicts the fold change in gene transcript level. The X axis indicates the sample type, either DWV-injected or PBS-injected. Student's t-test (P<0.05; t=2.637, df=22 & t=2.444, df=22 for respectively). Bars represent standard error. The star [★] above the bar denotes the 2^{-ΔΔct} calibrator. Each data point represents the mean of 12 determinations.

FIG. 7 depicts a graph of the temporal-based lipase activity in honey bees. Circles present data for newly emerged bees. Squares present data for 7-day-old bees. Up-pointing triangles present data for 14-day-old bees. Down-pointing triangles present data for 27-day-old bees. Significant differences between treatments were determined using linear regression (P<0.0001; F=8,127, DFn=3, DFd=952). Bars represent standard error. X axis indicates the time in minutes. Y axis depicts the measured Relative Fluorescent Units (RFU). Each data point represents the mean of 12 determinations.

FIG. 8 depicts a graph of the bee lipid levels through aging. The Y axis depicts the measured lipid levels in mg/mL. The X axis indicates the origin of the sample, either newly emerged bees, 7-day-old bees, 14-day-old bees, or 27-day-old bees. One-way ANOVA with Bonferroni's post hoc test was performed to assess significant differences (P<0.05; F=1.055, DFn=3, DFd=44). Bars represent standard error. Each data point represents the mean of 12 determinations.

FIG. 9 depicts a graph of a time course of lipase activity measured in control bees, or bees treated with sublethal doses of Imidacloprid. Circles present data for control bees. Squares present data for bees challenged with 5 ppb Imidacloprid. Up-pointing triangles present data for bees challenged with 50 ppb Imidacloprid. Statistical significance was determined using linear regression (P<0.01; F=4.612. DFn=2, DFd=1830). X axis indicates the time in minutes. Y axis depicts the measured Relative Fluorescent Units (RFU). Each data point represents the mean of 12 determinations.

FIG. 10 depicts a graph of the lipid levels in control bees, or bees treated with sublethal doses of Imidacloprid. The Y axis depicts the measured lipid levels in mg/mL. The X axis indicates the origin of the sample, either not challenged bees, bees challenged with 5 ppb Imidacloprid, or bees challenged with 50 ppb Imidacloprid. One-way ANOVA with Bonferroni's post hoc test was performed to assess significant differences (P<0.05; F=2.016, DFn=2, DFd=33). Bars represent standard error. Each data point represents the mean of 12 determinations.

### BRIEF DESCRIPTION OF THE SEQUENCES

The nucleotide sequences used in the instant disclosure, and their corresponding sequence identifiers are listed below in Table 1.

| Name | Sequence | SEQ ID NO: |
|---|---|---|
| DWV Sense | CGAAACCAACTTCTGAGGAA | 1 |
| Primer | | |
| DWV | TCGACAATTTTCGGACATCA | 2 |
| Antisense | | |
| Primer | | |
| Dorsal | TCGGATGGTGCTACGAGCGA | 3 |
| Forward | | |
| Primer | | |
| Dorsal | AGCATGCTTCTCAGCTTCTGCCT | 4 |
| Reverse | | |
| Primer | | |
| Apidaecin | TTTTGCCTTAGCAATTCTTGTTG | 5 |
| Forward | | |
| Primer | | |
| Apidaecin | GAAGGTCGAGTAGGCGGATCT | 6 |
| Reverse | | |
| Primer | | |

### DETAILED DESCRIPTION

The inventors have developed and refined a rapid high-throughput lipase enzyme activity assay for insects. The assay measures the number of lipid (fatty acid) molecules hydrolyzed per minute. The inventors surprisingly found that the measured lipase activity levels correlated directly with the insects' stress levels due to commonly encountered factors such as pathogens, pesticides, environmental change, and hypoxia.

Insects are any class of arthropods with a well-defined head, thorax, and abdomen. Adult insects have three pairs of legs, and typically one or two pairs of wings. Insects form the largest of the animal phyla, and are considered to be the most eminently successful group of all animals, with about 1 million described species. In some embodiments, the kits and methods of the invention are useful for measuring the lipase activity levels in Coleoptera, Lepidoptera, Hymenoptera, or Diptera. In some embodiments of the invention, the kits and methods taught here are useful for measuring the lipase activity levels in Hymenoptera. In some embodiments of the invention, the kits and methods taught herein are useful for measuring lipase activity levels in bees.

Lipases are an important group of biotechnologically-relevant enzymes. Lipases are found in food, dairy, detergent, and pharmaceutical industries. Lipases are also called triacylglycerol acyl hydrolases, and catalyze the hydrolysis of triacyl glycerides to release fatty acids and glycerol. Lipases catalyze esterification, interesterification, acidolysis, alcoholysis, and aminolysis. Lipases are useful in the synthesis of biopolymers and bio-diesel, the production of enantiopure pharmaceuticals, agrochemicals, and flavor compounds.

Different methods of measuring lipase activity are available in the art. Lipase activity can be measured by determining the rate of disappearance of the triglyceride substrate; the rate of production of fatty acids; or the rate of clarification of an emulsion. Lipase activity may be screened by directly observing changes in the appearance of a substrate; or by using titrimetric methods, colorimetric methods, fluorometric methods, turbidimetric methods, radioactive methods, conductance methods, or chromatographic methods, among others. Lipase activity may be assayed in crude or purified preparations. (See, for example, Thomson C. A. et al., 1999, “Detection and measurement of microbial lipase activity: a review,” Crit. Rev. Food Sci. Nutr., 39(2):165-187; Hasan F. et al., 2009, “Methods for detection and characterization of lipases: A comprehensive review,” Biotechnol. Adv. 27: 782-798).

Titrimetric methods of measuring lipase activity require little to no sophisticated equipment, and the analysis is straight-forward, but titrimetric methods tend to be time consuming. Colorimetric methods rely on the complexation of fatty acids in organic solvent with a divalent metal (usually copper), followed by spectrophotometric analysis of the metal in organic phase (copper-soap method). Colorimetric copper-soap methods are generally inexpensive, convenient, and reliable, but are less sensitive and may use toxic solvents. Fluorometric methods are sensitive, and allow continuous monitoring of the reaction kinetics.

Förster Resonance Energy Transfer is also referred to as Fluorescence Resonance Energy Transfer (FRET). FRET is a phenomenon where an energetically excited fluorophore (donor) transfers energy (not an electron) to another molecule (acceptor group) through a non-radioactive process through dipole-dipole coupling (through space). This photo-physical phenomenon was first established theoretically by Theodor Förster in 1948. Furthermore, the excited acceptor molecule returns to the ground state by losing its energy via photon emission (if the acceptor is a fluorophore, the energy is released as fluorescence).

The Marker Gene Technologies' (Eugene, Oreg., USA) fluorescent substrate 1,2-dioleoyl-3-(pyren-1-yl) decanoyl-rac-glycerol is commercially available for measuring lipoprotein lipase and hepatic triacylglycerol lipase. Duque and coworkers prepared fluorogenic and isomerically pure 1(3)-O-alkyl-2,3 (3,2)-diacyl glycerol compounds containing a fluorescent pyrene acyl chain as a quencher to determine lipase activity (Duque, M. et al., 1996, “New Fluorogenic triacylglycerol analogs as substrates for the determination and chiral discrimination of Lipase Activities,” J. Lipid Res. 37: 868-876). Mitnaul and coworkers used self-quenching BODIPY trygliceride and phophatidylcholine substrate analogs labeled with either one or two BODIPY dyes and introduced them into miscelles as a lipase activity test (Mitnaul, L. J., 2007, “Fluorogenic substrates for high-throughput measurements of endothelial lipase activity,” J. Lipid Res. 48: 472-482). As substrates useful in measuring lipase activity, Yang and co-workers prepared FRET substrates for lipases and esterases using an aliphatic 1,2-diol monoacylated with pyrenebutyric acid as a fluorophore, attached to a dinitropheylamino group as a quencher. Substrates were prepared with different branching, hydrophobicity, and length (Yang, Y. Z. et al., 2006, “Low background FRET substrates for lipases and esterases suitable for high-throughput screening under basic (pH 11) conditions,” Org. Biomol. Chem. 4: 1746-1754). Gupta an co-workers list additional lipase assays (Gupta, R. et al., 2003, “Lipase assays for conventional and molecular screening: an overview,” Biotechnol. Appl. Biochem. 37: 63-71).

EnzChek is a registered trade mark of Molecular Probes, Inc. (now owned by Invitrogen, a Thermo Fisher Scientific brand; Waltham, Mass., USA) for fluorescent chemicals and assay kits consisting primarily of a fluorescent reagent, reaction tubes, and reaction buffers for use in scientific research. The EnzChek lipase substrate currently available from Thermo Fisher Scientific under catalog No. E33955. The EnzChek molecular formula is C₅₈H₈₅BF₂N₆O₆, and the chemical structure found in the Thermo Fisher Scientific web site is shown on Structure I, below:

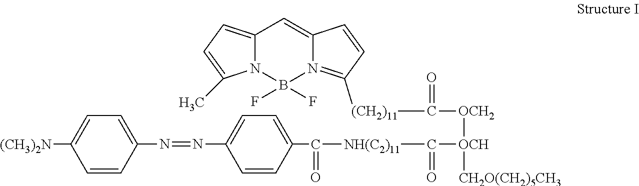

The EnzChek lipase substrate comprises a 4,4 difluoro-methyl-4-bora-3a,4a-diaza-s-indacene-3-dodecanoic acid (BODIPY) dye and a 4, 4 dimethylamino-phenyl-azo-benzoic acid (DABCYL acid) quencher in a triglyceride backbone. In EnzChek lipase substrate the fluorescent BODIPY-C12 FA derivative is in ester linkage at the ns-1 position of glycerol. The BODIPY-labeled substrate does not exhibit fluorescence in its unhydrolyzed state due to a bridged DABCYL quencher on the adjacent fatty acid arm. However, once the substrate is hydrolyzed by lipase, it releases a bright green BODIPY-labeled fatty acid.

Assays using EnzCheck lipase substrate to detect the activity of hepatic lipase (HL) and lipoprotein lipase (LPL) in mouse plasma have been described (Jin, W. U.S. Pat. No. 9,145,977, issued Sep. 29, 2015; and (Basu, D. et al., 2011 “Determination of lipoprotein lipase activity using a novelfluorescent lipase assay” J. Lipid Res. 52: 826-832). Andersen and Brask teach the synthesis and evaluation of fluorogenic triglycerides as lipase substrates and compare their activity with the lipase activity measured using the EnzChek lipase substrate. Three racemic fluorogenic triglycerides were assembled with Edans-DABCYL or a fluorescein-DABCYL FRET pair (Andersen, R. J. and Brask, J. 2016, “Synthesis and evaluation of fluorogenic triglycerides as lipase assay substrates,” Chem. Phys. Lipids 198: 72-79).

ZWITTERGENT detergents (trademark registered by Calbiochem-Behring Corp.; La Jolla, Calif., USA; last listed owner Merk KGaA, Darmstadt, Federal Republic of Germany) are synthetic zwitterionic detergents. Also known as sulfobetaines, ZWITTERGENTS offer the combined properties of ionic and non-ionic detergents, and retain their zwitterionic character over a broad pH range. This property is attributed to the presence of a quaternary ammonium ion and a sulfonate ion of equal strength. ZWITTERGENT detergents are available from octyl- to hexadecyl forms (C08; C10; C12; C14; and C16).

The kits and methods of the present invention are improvements over traditional lipase assay kits and methods. Compared to traditional kits and methods for measuring lipase activity, the kits and methods of the present invention are more sensitive, faster, and do not use radioactive materials or materials that may harm the environment.

In an embodiment, the invention relates to kits comprising a fluorogenic triglyceride comprising a dye and a quencher, and a substrate reaction buffer comprising Zwittergent detergent in PBS. The kits of the invention do not contain sodium chloride or Tris(hydroxymethyl)aminomethane hydrochloride.

Any fluorescent dye molecule may be attached to the triglyceride backbone. For example, the dye molecule may be a boron-dipyrromethene dye (BODIPY fluorescent dye); an ALEXA FLUOR dye; a PACIFIC GREEN dye; an OREGON GREEN dye (these four are trademarks or registered trademarks of Thermo Fisher Scientific, Waltham, Mass., USA); fluorescein; fluorescein isocyanate; tetrachlorfluorescein; a CAL fluor chemical dye such as Gold 540 or Orange 560 (trademarks of Biosearch Technologies, Inc., Novato, Calif., USA); 4,5-dichloro-dimethoxy-fluorescein; hexachloro-fluorescein; Evans Blue; or DYLIGHT fluorescent dye (Pierce Biotechnology, Rockford, Ill., USA).

Any quenching molecule may be attached to the triglyceride backbone. For example, the quenching molecule may be a Black Hole Quencher (trademark of Biosearch Technologies, Inc. Petaluma, Calif., USA), or a 4-4′-(dimethylaminophenylazo) benzoic acid (DABCYL acid).

In some embodiments of the invention, the fluorogenic triglyceride in the kit comprises a BODIPY dye, and a DABCYL acid quencher. In some embodiments of the invention, the fluorogenic triglyceride comprising a dye and a quencher has the chemical structure set forth in Structure I.

The inventors have developed and refined a rapid high-throughput assay to determine the lipase activity levels in insects. The inventors have surprisingly found that the lipase activity levels measured using the developed lipase activity assay may be used for the general detection of honey bee (Apis mellifera) stress. The assay measures the number of lipid (fatty acid) molecules hydrolyzed per minute, a phenomenon that is directly correlated to several different stressors such as pathogens, pesticides, environmental change, aging, and hypoxia that honey bees commonly encounter.

The inventors have demonstrated that the kits and methods taught herein are useful for the measurement of lipase activity in the European honey bee. The method of measuring lipase activity in honey bees taught herein displayed sensitivity similar to or better then methods known in the art for measuring lipase activity such as those taught above, and those taught by Hasan F. et al. (2009, “Methods for detection and characterization of lipases: A comprehensive review,” Biotechnol. Adv. 27: 782-798).

Samples of healthy European honey bees, Apis mellifera, were collected from colonies maintained in the apiaries of the USDA-ARS Bee Research Laboratory in Beltsville, Md., USA. All treatment of honeybees conformed with the laws of the USA in relation to the care and use of laboratory animals. The healthy colonies were confirmed free of Varroa infestation and had no detectable pathogens based on a monthly survey for parasites and pathogens. Worker pupae sourced at the white eye stage (12th-13th days of development) received injections into the hemolymph using a syringe with a 0.3 mm outer diameter needle with either 10 μL PBS or with about 10⁷ or about 10⁸ DWV in PBS. After injection, the pupae were kept in an incubator at 33° C. and 80% relative humidity for 24 hours before harvesting. To measure the lipase activity, each injected pupa was homogenized in 1 mL 4×PBS. A 100 μL aliquot of homogenized pupae was diluted with 200 μL 4× sample buffer (0.015 g/mL BSA; 0.6 mg/mL Zwittergent 3-16; 4×PBS). Twenty μL of each diluted homogenized pupae was pipetted into a well of a 96-well plate, followed by the addition of 80 μL EnzChek working solution (Reaction buffer containing between 0 μM and 20 μM of EnzCheck lipase substrate in DMSO).

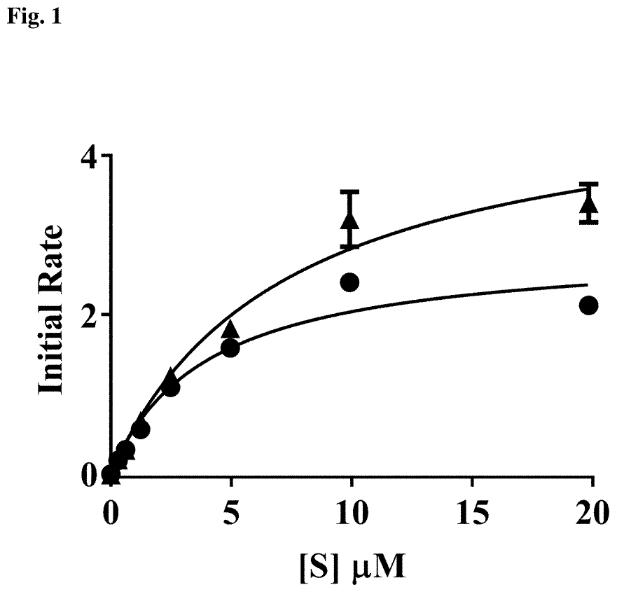

Lipase activity was measured fluorescently with an excitation/emission peak at 482/515 nm using a BioTek Synergy H1 Hybrid spectrophotometer (Winooski, Vt., USA) at 37° C. A plot of the initial rates against substrate concentrations is shown in FIG. 1, where data for PBS injected pupae is shown with triangles and data for DWV-injected pupae is shown with circles. Non-linear regression fit was performed using the Michaelis-Menten equation. The P value refers to a comparison between the data obtained with PBS-injected pupae and the data obtained with WDV-injected pupae (P<0.0001). Each data point represents the mean of 12 determinations. The Michaelis-Menten constant (Kₘ) for PBS-injected pupae was 4.03 μM, and for DWV-injected pupae was 7.28 μM. The maximum reaction velocity (Vₘₐₓ) for PBS-injected pupae was 2.87 μM/minute, and for DWV-injected pupae was 4.89 μM/minute. Thus, a significant difference was observed (of approximately 1.75-fold) in both Kₘ, and Vₘₐₓ for DWV-injected individuals as compared to PBS-injected individuals. After assessing the cost of each assay, it was determined that the lowest concentration of EnzChek lipase substrate suitable for detecting significant differences between healthy and DWV infected bees was 2.86 μM.

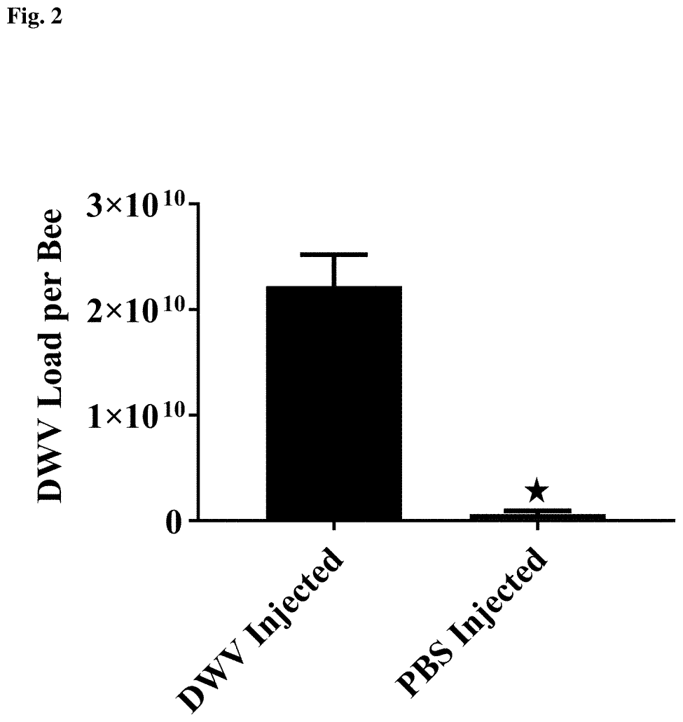

As expected, the inventors determined that higher initial levels of lipase activity measured using the EnzChek lipase substrate and the buffers taught herein correlate with higher DWV RNA levels. These results suggest that the methods taught here for determining lipase activity in bees are useful for determining DWV infection levels. RNA was extracted from pupae twenty four hours post-injection with PBS or DWV in PBS and the DWV titers were determined using real time quantitative PCR (qPCR). Fluorescence values were determined, and amplification plots were generated by the Mx4000 System software. The concentration of DWV in honeybees was analyzed by using the comparative ΔΔCt method. The qPCR results were expressed as means±standard deviations for Ct values for DWV-injected or PBS-injected pupae. For the comparative Ct method to be valid, eight threefold serial dilutions (1,000, 333, 111, 37, 12, 4, 1.37, and 0.45 ng) of a total RNA sample were used for RT-PCR amplification to confirm that the amplification efficiencies of the DWV and 3-actin RT-PCRs were similar. The equations for relative standard curves and relative efficiency plots were calculated by using the Statistix7 statistical software (Analytic Software, Tallahassee, Fla., USA). The virus concentrations of all samples tested were normalized by subtracting the Ct value of β-actin from the Ct value of DWV. The fold differences in the concentrations for the different pupae were also calculated. As seen in FIG. 2, the DWV load in DWV-injected pupae measured over 2×10¹⁰; while the DWV load in PBS-injected pupae was close to the detection level.

The levels of lipase activity measured in bee pupae injected with DWV or PBS using EnzChek lipase substrate and the buffers taught herein indicated that measured lipase activity levels may be used to diagnose bee stress due to an active DWV infection.

To determine if it is possible to detect a difference between healthy bees and DWV-infected bees using the EnzChek lipase substrate and the buffers taught herein, a time course assay was performed using a set amount of EnzChek lipase substrate and a fixed amount of extracted pupae. Briefly, 200 μL of sample buffer were added to 100 μL of healthy pupae or DWV-infected pupae homogenized in PBS, followed by the addition of 80 μL EnzChek lipase substrate working solution containing 2.48 μM EnzChek lipase substrate in DMSO.

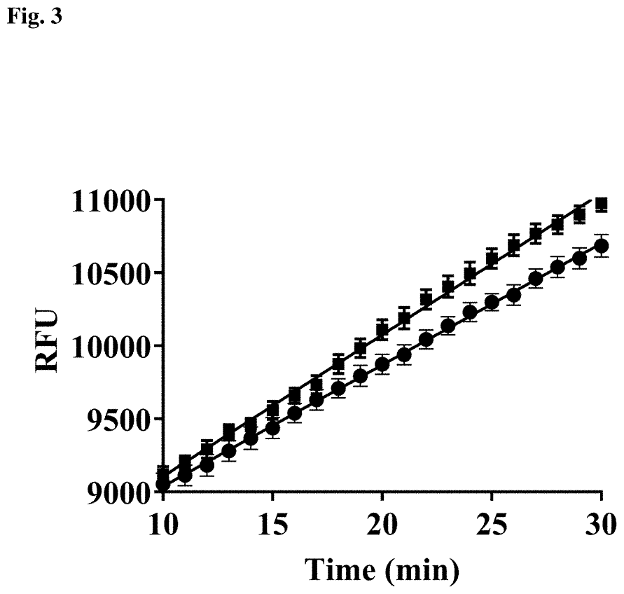

Fluorescence, as an indication of lipase activity, was measured with an excitation/emission peak at 482/515 nm using a BioTek Synergy H1 Hybrid spectrophotometer (Winooski, Vt., USA), and the data is presented in FIG. 3. In this figure, the relative fluorescence intensity (RFU) measured was plotted against time. Using linear regression, a significant difference in slopes was observable between individuals from DWV-infected hives, and individuals from healthy hives. The slope obtained for DWV-infected individuals was about 13.8 higher than the slope obtained for healthy individuals. Linear regression results show a significant difference between data obtained from bees from DWV-infected hives and bees from healthy hives (P<0.0001). This data indicates a significant increase in fatty acid liberation in DWV-infected individuals when compared to the results obtained for healthy individuals.

In an embodiment, the invention relates to a method for determining levels of lipase activity in an insect biological sample. The method comprises mixing an insect biological sample in PBS with a working solution comprising a fluorogenic triglyceride comprising a dye and a quencher, DMSO, C16 Zwittergent, and PBS; and measuring the emitted fluorescence, where the measured emitted fluorescence is an indication of the LPL activity in the insect biological sample. In some embodiments of the invention, the LPL activity is measured in a biological sample from a Coleoptera, a Lepidoptera, a Hymenoptera, or a Diptera. In some embodiments of the invention the insect biological sample where the LPL activity is measured is homogenized egg, homogenized larva, homogenized pupa, or homogenized adult. In some embodiments of the invention, the insect biological sample where the LPL activity is measured is Hymenoptera. In some embodiments of the invention, the Hymenoptera where the LPL activity is measured is a bee. In some embodiments of the invention, the insect biological sample is from a drone bee, a worker bee, or a queen bee.

In an embodiment, the invention relates to a method for determining the levels of lipase activity in a insect biological sample, comprising adding a portion of the insect biological sample in PBS to a well of a 96-well plate; adding working solution comprising a fluorogenic triglyceride comprising a dye and a quencher, DMSO, Zwittergent, and 1×PBS to each well of the 96-well plate containing insect biological sample in PBS; and measuring the fluorescence with a 482 nm excitation/515 nm emission.

Currently, overt viral infection levels are detected and quantified using real-time RT-qPCR; a process that is relatively expensive in reagents and time (hours to days). The lipase-based approach taught here requires only live bees and fluorescent lipid substrate, and can be carried out to completion and analyzed within the span of an hour. The likely cost per sample is between one and two orders of magnitude less than that of current PCR-based methods. The invention incorporates several novel and beneficial features that include: ease of use, cost reduction, and increased sampling size. In terms of cost benefit analysis, each 96-well plate can assay approximately 48-96 hives of pooled bees (1 mL sample buffer per bee), leading to analyzing approximately 192-384 hives per US$236 plus labor and time.

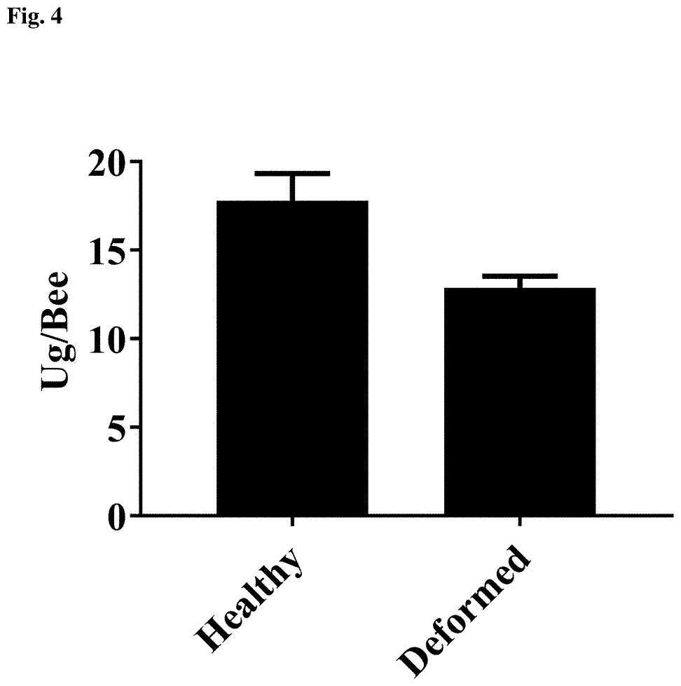

The inventors surprisingly found that the lipase activity levels measured in healthy and DWV-infected hives correlate with the lipid levels measured in the bees using the vanillin assay. As can be seen on FIG. 4, the lipid levels in samples from DWV-infected bees (deformed) were below 15 μg/bee, while the lipid levels in samples from healthy bees were above 15 μg/bee. These results are consistent with the lipase activity results reported in Example 1, where the measured lipase activity in pupae injected with DWV was higher than the measured lipase activity in healthy bees. This serves as evidence that the mechanics of viral stress are capable of inducing lipase activity that leads to reduced total lipids. Furthermore, this is indicative of the ability of the lipase assay using EnzChek lipase substrate and the buffers taught in Example 1 to directly detect biotic stress. The total lipid levels in the DWV-infected bees were reduced approximately one-fold when compared to the total lipid levels in healthy bees (Student's t-test P<0.007). Bars represent standard error.

The inventors surprisingly found that it is possible to distinguish between terminal hives and healthy hives by measuring the lipase activity levels in the bees using the methods taught in the instant application. Field-caught adult bees were obtained by observing symptomatic hives and collecting adults directly from the brood frame via vacuum. Bees gathered from field hives were divided into three groups: healthy, asymptomatic (healthy-looking bees collected from hives with observable terminal or deformed bees), and deformed. Total RNA was obtained from bees using TRIzol reagent for isolating biological material from organic tissue (Molecular Research Center, Inc., Cincinnati, Ohio, USA) following the manufacturer's specifications (Invitrogen, Carlsbad, Calif., USA), and RNA was synthesized into cDNA using superscript III first-strand synthesis system per manufacturer's instructions (Invitrogen, Carlsbad, Calif., USA).

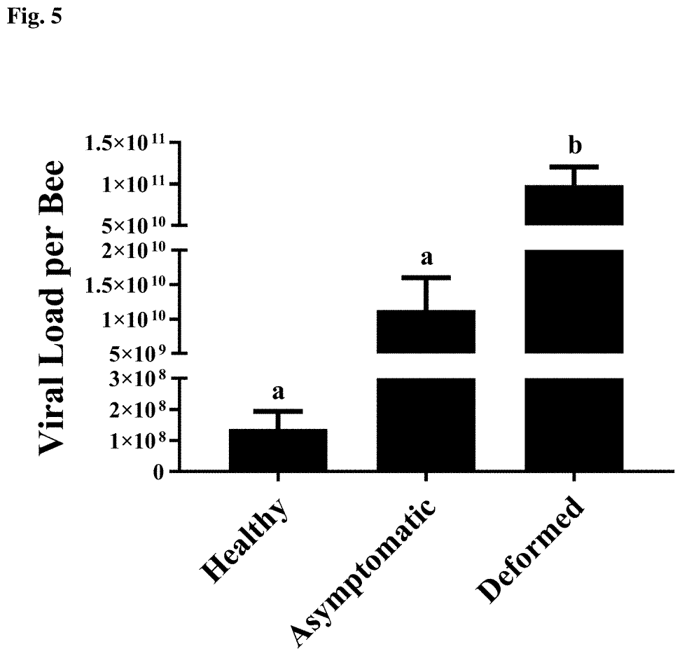

The DWV viral levels were then ascertained using qPCR on a Bio-Rad CFX 384 Touch Real-Time PCR Detection System (Hercules, Calif., USA). As seen in FIG. 5, virus-infected bees exhibiting deformed or poorly developed wings contained about 1×10¹¹ viral load per bee; while asymptomatic bees contained about 1×10¹⁰; and healthy bees contained about 1×10⁸ viral load per bee. A one-way ANOVA with a post hoc Tukey test was used to measure significance (P<0.0001 between a & b groups). Bars represent standard error. The virus-infected bees exhibiting deformed or poorly developed wings presented with about 1000-fold increase in viral levels when compared to healthy bees. The asymptomatic virus-infected bees displayed a non-significant 100-fold difference compared to healthy bees.

Inducible antiviral barriers under the control of NF-1B or JAK-STAT control appear to be targeted in virally-infected insects instead of the RNAi-mediated mechanisms. The apidaecin gene encodes a proline-rich antimicrobial peptide under NF-1B transcriptional control. Following conformational changes of the activated NF-1B receptor, the NF-1B transcription factor Dorsal is translocated to the nucleus. Di Prisco found a positive correlation between the transcription rate of apidaecin and the number of DWV genome copies in bees (see Di Prisco, G, et al. 2016). Thus, the relative differential expression of Dorsal and Apidaecin are useful markers for the bee's immune response.

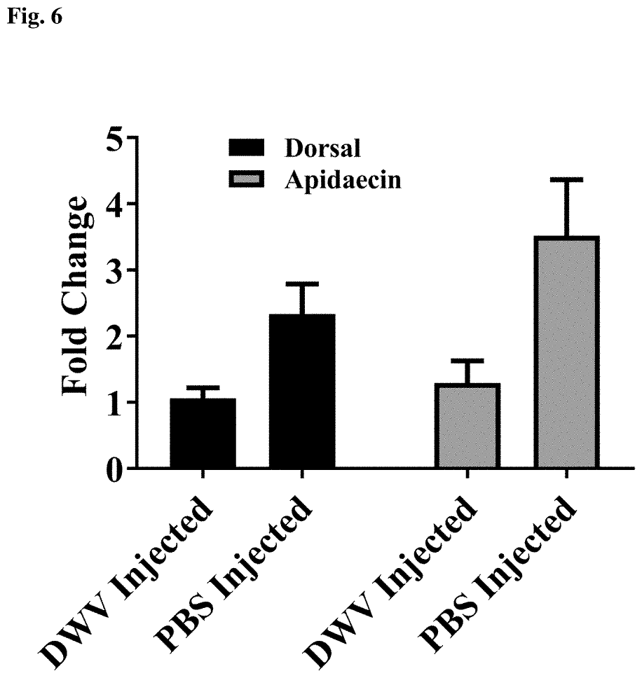

The inventors confirmed that DWV injection adversely affected the bee's immune response by determining the relative differential expression of Dorsal and Apidaecin in DWV-injected bees as compared to PBS-injected bees. The transcript levels of Dorsal and Apidaecin were measured 24 hours after injection, and the amount of change from non-injected was calculated using the comparative 2^{-ΔΔct} method. FIG. 6 shows that the mRNA fold change of Dorsal and Apidaecin in DWV-injected pupae was about 1; the fold change of Dorsal in PBS-injected pupae was about 2; and the fold change of Apidaecin was about 3. These results indicate that the mRNA transcript levels for both, Dorsal and Apidaecin, were significantly reduced in DWV-injected pupae when compared to PBS-injected pupae. Dorsal, a NF-1B transcription factor, was reduced approximately 2.5-fold after injection. Apidaecin, an anti-microbial peptide, typically transcribed after infection, was reduced approximately 3.5-fold after injection.

Among the facultatively sterile female bee workers, there is an age-correlated behavioral division of labor, referred to as temporal polyethism. Young workers perform brood-nest associated tasks such as brood-cell cleaning and larval feeding. Middle-aged bees typically perform food processing, nest construction, and guarding. Finally, older bees progress to foraging outside the nest for food (Siegel, A. J. et al., 2013, “In-hive patterns of temporal polyethism in strains of honey bees (Apis mellifera) with distinct genetic backgrounds,” Behav. Ecol. Sociobiol. 67: 1623-1632).

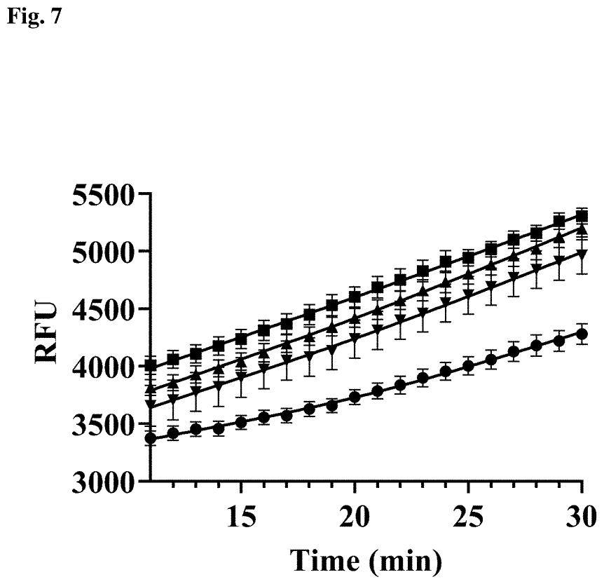

The inventors surprisingly found that using the methods taught herein to determine the lipase activity levels in bees it is possible to accurately detect a correlation between fat metabolism and bee aging. Using the methods disclosed in Example 3, the lipase activity was measured in newly emerged bees, 7-day-old bees (nurses), 14-day-old bees (transitioning to forager), and 27-day-old bees (forager). The linear range and slopes of the measured lipase activities were graphed and are shown in FIG. 7. Newly emerged bees appeared to have the lowest lipase activity measured and lowest slope of the measured lipase activity when compared to the other bee stages. The measured lipase activity and the lipase activity slope obtained for nurse bees, bees transitioning to forager, and forager bees appeared to be very similar to each other.

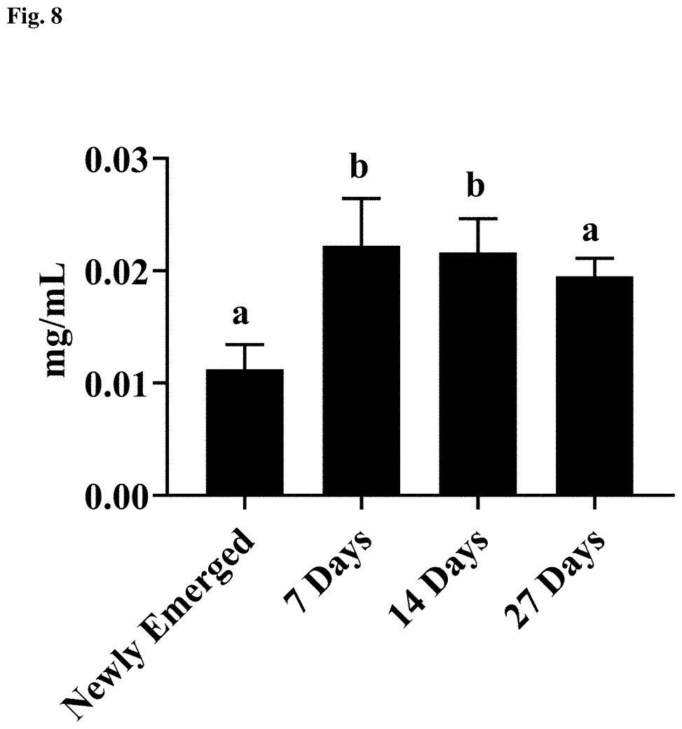

Similarly, the inventors surprisingly found that the lipid content at different stages of bee aging correlates with the fat metabolism in the bees. The lipid levels in newly emerged bees, 7-day-old bees, 14-day-old bees, and 27-day-old bees were determined using the vanillin assay. The lipid levels measured during honey bee aging are depicted in FIG. 8. As detected using the vanillin assay, lipid levels were lowest for newly emerged bees, and were almost twice as much for 27-day-old bees. Nurse bees (7-day-old) bees and 14-day-old bees had similar lipid levels, which were slightly higher than those measured for the 27-day-old bees. One-way ANOVA with Bonferroni's post hoc test was performed to assess significant differences (P<0.05). Bars represent standard error. These results are in agreement with the measured lipase activity shown in FIG. 7, where the lowest lipase activity measured was for newly emerged bees, and the lipase activity measured for nurse bees, bees transitioning to forager, and forager bees appeared to be very similar to each other.

Neonicotinoid insecticides are synthetic derivatives of nicotine, an alkaloid compound found in the leaves of many plants in addition to tobacco, and are toxic to insects. Nicotinoids can target several pests in the Homoptera, Coleoptera, and Lepidoptera families. Nicotinoids have been under scrutiny due to their persistence in the soil, ability to leach into the environment, high water solubility, and potential negative health implications for non-target organisms such as pollinators. Imidacloprid is a neonicotinoid insecticide in the chloronicotinyl nitroguanidine chemical family. The IUPAC name is 1-(6-chloro-3-pyridylmethyl)-N-nitroimidazolidin-2-ylideneamine, and the CAS registry number is 138261-41-3. Imidacloprid is used to control sucking insects, termites, some soil insects, and fleas on pets. It has been used in products sold in the United States since 1994. Imidacloprid disrupts the ability of the nerves to send signals, and the nervous system stops working the way it should. Imidacloprid is much more toxic to insects and other invertebrates than it is to mammals and birds because it binds better to the receptors of insect nerve cells than it does to the mammal and bird receptors. Imidacloprid is a systemic insecticide, which means that plants take it up from the soil or through the leaves and it spreads throughout the plants' stems, leaves, fruit, and flowers. Insects that chew or suck on the treated plants end up eating the imidacloprid as well. Once the insects eat the imidacloprid, it damages their nervous system and they eventually die. Even at extremely low doses, neonicotinoids are lethal to honey bees and other beneficial insects (Li Z., et al., 2017, “Differential physiological effects of neonicotinoid insecticides on honey bees: A comparison between Apis mellifera and Apis cerana,” Pestic. Biochem. Phys. 140: 1-8).

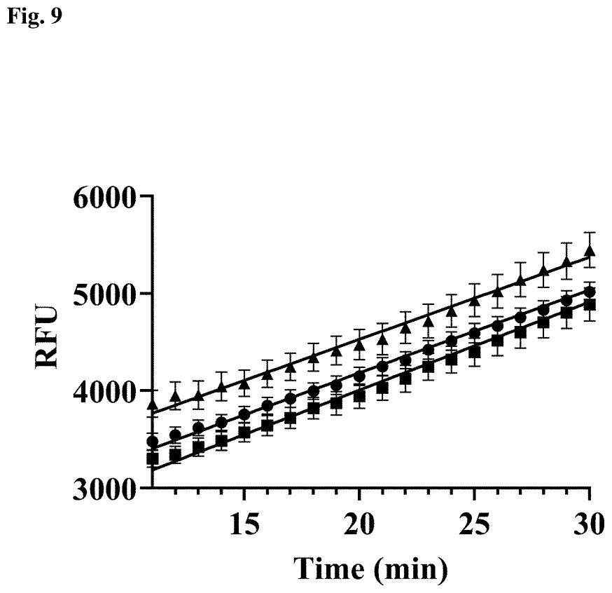

The inventors surprisingly found that the lipase activity levels measured using EnzChek lipase substrate and the buffers taught herein, accurately correlate to the bees' stress derived from pesticides. Healthy honey bees were challenged in a cup-cage environment with either 5 ppb or 50 ppb of the neonicotinoid imidacloprid. The lipase activity levels were measured during a 50 minute time course, as taught herein. A graph of the measured RFU plotted against time is shown in FIG. 9. The measured lipase activity levels indicated that bees receiving 5 ppb or 50 ppb imidacloprid displayed a dose-dependent response to the pesticide. While the slopes of the graphed lipase activity levels were similar for control bees, and bees challenged with 5 ppb, the lipase activity levels of bees challenged with 50 ppb imidacloprid were significantly higher.

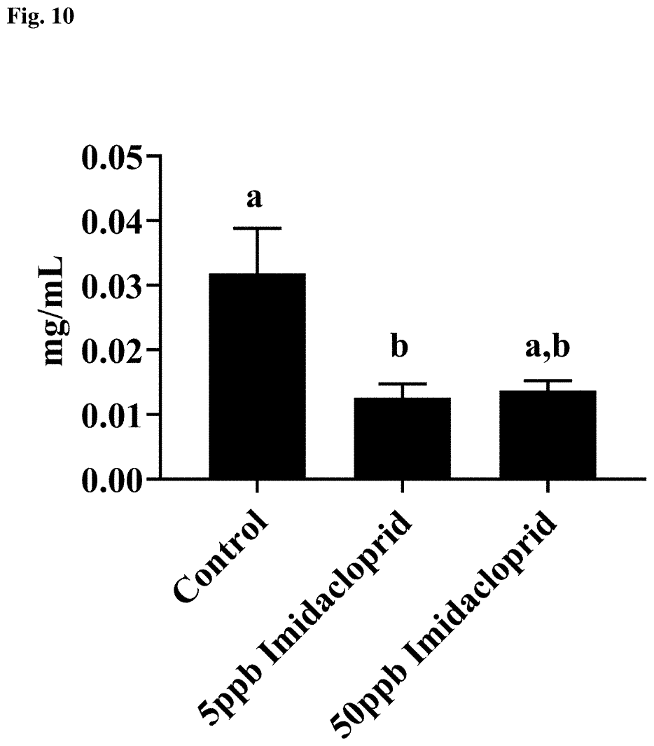

The inventors surprisingly found that the lipase activity levels measured using EnzChek lipase substrate and the buffers taught herein correlate with the total lipid content of pesticide-treated bees, accurately predicting and detecting the bees' pesticide-derived stress. The total lipid content on bees challenged with either 5 ppb or 50 ppb imidacloprid was measured using the vanillin assay, performed as in Example 4. The measured lipid levels (in mg/mL) were plotted, and the results are shown on FIG. 10. The total lipid content for bees treated with 5 ppb imidacloprid (P<0.01) or 50 ppb imidacloprid (P<0.05) was approximately 3-fold lower than the total lipid content measured in control bees.

In an embodiment, the invention relates to a kit for determining the levels of lipase activity in an insect. In some embodiments of the invention, the kit for determining the levels of lipase activity in an insect comprises a sample buffer comprising bovine serum albumin (BSA) and C16 Zwittergent detergent in PBS; a substrate reaction buffer comprising C16 Zwittergent detergent in PBS; and a fluorogenic triglyceride comprising a dye and a quencher in DMSO. In some embodiments of the invention, in the kit for determining the levels of lipase activity in an insect, the substrate reaction buffer and the fluorogenic triglyceride comprising a dye and a quencher in DMSO are in one container. In some embodiments of the invention, in the kit for determining the levels of lipase activity in an insect, the sample buffer, the substrate reaction buffer, and the fluorogenic triglyceride comprising a dye and a quencher in DMSO are in separate containers.

In order to facilitate review of the various embodiments of this disclosure, the following explanations of specific terms are provided:

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below.

As used herein, “fat burning” refers to the release of fats by the hormone-sensitive lipase (HSL). Fats are stored as triglycerides in fat body cells and are released via the activity of HSL.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

The foregoing detailed description and certain representative embodiments and details of the invention have been presented for purposes of illustration and description of the invention. It is not intended to be exhaustive or to limit the invention to the precise forms disclosed. It will be apparent to practitioners skilled in the art that modifications and variations may be made therein without departing from the scope of the invention.

All publications, patents, and patent applications mentioned in this specification are herein incorporated by reference to the same extent as if each individual publication, patent, or patent application was specifically and individually indicated to be incorporated by reference. In addition, the materials, methods, and examples are illustrative only and not intended to be limiting.

### EXAMPLES

Having now generally described this invention, the same will be better understood by reference to certain specific examples, which are included herein only to further illustrate the invention and are not intended to limit the scope of the invention as defined by the claims.

### Example 1

### Lipase Enzymatic Kinetics Determination

This Example demonstrates that the EnzCheck Lipase substrate obtained from Invitrogen (Carlsbad, Calif., USA) together with the buffers taught herein are useful for the measurement of lipase activity in the European honey bee (Apis mellifera logustica). The measured lipase activity levels indicate that after viral infection the levels of fat metabolism differ for immature honey bees.

The commercially available EnzChek lipase substrate (Invitrogen, Carlsbad, Calif., USA) is composed of a 4,4 difluoro-methyl-4-bora-3a,4a-diaza-s-indacene-3-dodecanoic acid (BODIPY) dye and a [4-((4-dimethylamino) phenyl)azo)benzoic acid] (DABCYL acid) quencher in a triglyceride backbone of Formula I. The EnzChek lipase substrate has Thermo Fisher Scientific (Waltham, Mass., USA) Catalog No. E33955. Ten× (10×) premixed phosphate buffered saline (PBS) pH 7.2 solution, having catalog No. 11666789001, was obtained from Roche (Basel, Switzerland). Fatty acid-free bovine serum albumin (BSA), having catalog No. A7906-100 mg, was obtained from Sigma (St. Louis, Mo., USA). Zwittergent 3-16, having catalog No. EMD-693023-5g, was obtained from EMD Millipore (Burlington, Mass., USA). Dimethyl Sulfoxide (DMSO), having catalog No. E33955, was obtained from Thermo Fisher (Waltham, Mass., USA). Clear polystyrene 96-well plates, having catalog No. 3631, were obtained from Corning (Corning, N.Y., USA).

To prepare 4× Sample buffer 0.6 g BSA; 24 mg Zwittergent detergent; and 16 mL 1× PBS were added to 40 mL deionized water. One thousand μM EnzChek lipase substrate was prepared by adding 100 μL DMSO to 100 μg EnzCheck lipase substrate. EnzChek Lipase substrate reaction buffer was prepared by dissolving 25 mg Zwittergent 3-16 detergent in 50 mL PBS. EnzChek working solution was prepared by adding EnzChek lipase substrate to 8.5 mL EnzChek Lipase substrate reaction buffer.

Samples of healthy European honey bees, Apis mellifera, were collected from colonies maintained in the apiaries of the USDA-ARS Bee Research Laboratory in Beltsville, Md., USA. All treatment of honeybees conformed with the laws of the USA in relation to the care and use of laboratory animals. The colonies were surveyed for parasites and pathogens monthly following methods described by Di Prisco, G., et al., 2011, “Dynamics of persistent and acute deformed wing virus infections in honey bees, Apis mellifera,” Viruses 3: 2425-2441. Frames with sealed brood were removed from the colony, and pupae were individually removed from brood cells for later injection.

The DWV virus was propagated in infected honey bee pupae. For the preparation of extracts containing infectious DWV particles, individual pupae were homogenized with 1 mL PBS, clarified by centrifugation at 3000 g for 5 minutes, followed by filtration through a 0.22 μM mesh filter (Posada-Florez F., et al., 2019, “Deformed wing virus type A, a major honey bee pathogen, is vectored by the mite Varroa destructor in a non-propagative manner,” Sci. Rep. 9:12445). The DWV concentrations in each extract were quantified using RT-qPCR following Chen, Y. P., et al. (2005, Quantitative Real-Time Reverse Transcription-PCR Analysis of Deformed Wing Virus Infection in the Honeybee (Apis mellifera L.),” Appl. Environ. Microbiol. 71(1): 436-441). Briefly, DWV RNA was amplified using the Access RT-PCR system (Promega, Madison, Wis., USA), DWV-sense primer (CGAAACCAACTTCTGAGGAA; set forth in SEQ ID NO: 1), and DWV-antisense primer (GTGTTGATCCCTGAGGCTTA; set forth in SEQ ID NO: 2) following the manufacturer's instructions. The amplified products were quantified using a Bio-Rad CFX-384 Real Touch (Hercules, Calif., USA).

Worker pupae sourced at the white eye stage (12th-13th days of development) received injections into the hemolymph using a syringe with a 0.3 mm outer diameter needle with either 10 μL PBS or with about 10⁷ DWV in PBS. After injection, the pupae were kept in an incubator at 33° C. and 80% relative humidity for 24 hours before harvesting. To measure the lipase activity, each injected pupae was homogenized in 1 mL 4×PBS. A 100 μL aliquot of homogenized pupae was diluted with 200 μL 4× sample buffer (0.015 g/mL BSA; 0.6 mg/mL Zwittergent 3-16; 4×PBS). Twenty μL of each diluted homogenized pupae was pipetted into a well of a 96-well plate, followed by the addition of 80 μL EnzChek working solution (Reaction buffer containing between 0 μM and 20 μM of EnzCheck lipase substrate in DMSO).

Lipase activity was measured fluorescently with an excitation/emission peak at 482/515 nm using a BioTek Synergy H1 Hybrid spectrophotometer (Winooski, Vt., USA) at 37° C. A plot of the initial rates against substrate concentrations is shown in FIG. 1, where data for PBS injected pupae is shown with triangles and data for DWV-injected pupae is shown with circles. Non-linear regression fit was performed using the Michaelis-Menten equation. The initial rate was plotted on the y-axis and reflects reaction velocity compared to increasing concentrations of the fluorescent substrate, ergo, a lower curve equates to higher lipase activity. The P value refers to a comparison between the data obtained with PBS-injected pupae and the data obtained with WDV-injected pupae (P<0.0001). Each data point represents the mean of 12 determinations. The Michaelis-Menten constant (Kₘ) for PBS-injected pupae was 4.03 μM, and for DWV-injected pupae was 7.28 μM. The maximum reaction velocity (Vₘₐₓ) for PBS-injected pupae was 2.87 μM/minute, and for DWV-injected pupae was 4.89 μM/minute. Thus, a significant difference was observed (of approximately 1.75-fold) in both Km, and Vmax for DWV-injected individuals as compared to PBS-injected individuals.

The results in this example show that EnzChek lipase substrate with the buffers taught herein are suitable for the measurement of lipase activity in immature honey bees. The results provided in this example suggest that the levels of fat burning in immature honey bees change after DWV infection. These measurements adequately monitor viral infection stress in honey bees with sensitivity similar to that of previous methods.

### Example 2

### Correlation of Lipase Activity with Active DWV Infection

This Example demonstrates that the levels of lipase activity measured using EnzChek lipase substrate and the buffers taught herein may be used to diagnose an active DWV infection.

To determine if the levels of lipase activity measured using EnzChek lipase substrate and the buffers taught herein correlated with active DWV-induced infection, RNA was extracted from pupae twenty four hours post-injection with PBS or DWV in PBS and the DWV titers were determined using real time quantitative PCR (RT qPCR). To normalize the results, RT-qPCR was performed under the same conditions on the A. mellifera β-actin. Amplification was carried out using the Access RT-PCR system (Promega, Madison, Wis., USA) following the manufacturer's instructions with the primers disclosed above, and a Bio-Rad CFX-384 Real Touch (Hercules, Calif., USA).

A DWV-negative template control, a DWV-positive template control, and a no-template (H₂O) control were included in each reaction run. The amplification results were expressed as the threshold cycle (Ct) value, which represented the number of cycles needed to generate a fluorescent signal greater than a predefined threshold, using a Bio-Rad CFX-384 Real Touch (Hercules, Calif., USA).

The concentration of DWV in honeybees was analyzed by using the comparative absolute Ct quantification compared to DWV Cts of known concentration. The RT-qPCR results were expressed as means±standard deviations for Ct values for DWV-injected or PBS-injected pupae. For the absolute Ct method to be valid, six, 10-fold serial dilutions of known DWV standard was compared to the Ct values of the PBS or DWV injected pupae. As seen in FIG. 2, the DWV load in DWV-injected pupae measured over 2×10¹⁰; while the DWV load in PBS-injected pupae was close to the detection level.

The results in this example indicate that injecting DWV using the method taught above was effective at inducing an active infection, and the lipase activity measured using EnzChek lipase substrate together with the buffers taught herein correlates with the bee's stress.

### Example 3

### Response of Enzchek Lipase Substrate

This Example demonstrates that the EnzChek lipase substrate together with the buffers taught herein are an economical reagent useful for detecting significant differences between healthy and stressed bees.

To determine if it is possible to detect a difference between healthy bees and DWV-infected bees using the EnzChek lipase substrate and the buffers taught herein, a time course assay was performed using a set amount of EnzChek lipase substrate and a fixed amount of extracted pupae. Briefly, 200 μL of sample buffer were added to 100 μL of healthy pupae or DWV-infected pupae homogenized in PBS, followed by the addition of 80 μL EnzChek lipase substrate working solution containing 2.48 μM EnzChek lipase substrate in DMSO. Fluorescence, as an indication of lipase activity, was measured with an excitation/emission peak at 482/515 nm using a BioTek Synergy H1 Hybrid spectrophotometer (Winooski, Vt., USA), and the data is presented in FIG. 3. In this figure, the relative fluorescence intensity (RFU) measured was plotted against time. Using linear regression, a significant difference in slopes was observable between individuals from DWV-infected hives, and individuals from healthy hives. The slope obtained for DWV-infected individuals was about 13.8 higher than the slope obtained for healthy individuals. Linear regression results show a significant difference between data obtained from bees from DWV-infected hives and bees from healthy hives (P<0.0001). This data indicates a significant increase in fatty acid liberation in DWV-infected individuals when compared to the results obtained for healthy individuals.

This Example proposes that the methods described herein using EnzChek lipase substrate with the buffers disclosed Example 1 is a cost-effective method to measure bee stress. Using one container of 100 μg EnzChek, obtained from ThermoFisher Scientific for US$236 (in September 2019) allows for the measurement of the lipase activity levels in detection of stress levels about 384 bee samples (Four 96-well plates).

### Example 4

### Correlation of Lipase Activity with Lipid Levels

This Example demonstrates that the lipase activity levels measured using EnzChek lipase substrate and the buffers taught herein in healthy and DWV-infected hives correlate with the lipid levels measured in healthy and DWV-infected hives using the vanillin assay.

To determine if the lipase activity measured in healthy and DWV-infected hives correlated with the measured lipid levels, the vanillin assay (Foray, V., et al., 2012, “A handbook for uncovering the complete energetic budget in insects: the van Handel's method (1985) revisited,” Physiol. Entomol. 37: 295-302) was used to measure the lipid levels in the bees. Briefly, vanillin reagent was prepared by mixing vanillin (V1104; Sigma-Aldrich, St. Louis, Mo., USA) with 68% ortho-phosphoric acid, reaching a final concentration of 1.2 g/L. For the assay, 100 μL of homogenized pupae in PBS was transferred into a borosilicate microplate well and heated at 90° C. until complete solvent evaporation. Ten microliters of 98% sulphuric acid was then added to each well, and the microplate was incubated at 90° C. for 2 minutes in a water bath. After cooling the microplate on ice, 190 μL of vanillin reagent was added to each well. The plate was homogenized, incubated at room temperature for 15 minutes and its absorbance was measured spectrophotometrically at 525 nm.

As can be seen on FIG. 4, the lipid levels in samples from DWV-infected bees (deformed) were below 15 μg/bee, while the lipid levels in samples from healthy bees were above 15 μg/bee. These results are consistent with the lipase activity results reported in Example 1, where the measured lipase activity in pupae injected with DWV was higher than the measured lipase activity in healthy bees. This serves as evidence that the mechanics of viral stress are capable of inducing lipase activity that leads to reduced total lipids. Furthermore, this is indicative of the ability of the lipase assay using EnzChek lipase substrate and the buffers taught in Example 1 to directly detect biotic stress. The total lipid levels in the DWV-infected bees were reduced approximately one-fold when compared to the total lipid levels in healthy bees (Student's t-test P<0.007). Bars represent standard error.

The results in this example indicate that the levels lipase activity measured using EnzChek lipase substrate and the buffers taught in Example 1 correlate with the levels of lipids measured using the vanillin assay.

### Example 5

### Distinguish Between Symptomatic and Healthy Hives

This Example demonstrates that it is possible to distinguish between symptomatic hives and healthy hives using the method taught in Example 3 to determine the level of lipase activity in the bees.

Field-caught adult bees were obtained by observing symptomatic hives and collecting adults directly from the brood frame via vacuum. Bees gathered from field hives were divided into three groups: healthy, asymptomatic (healthy-looking bees collected from hives with observable terminal or deformed bees), and deformed. Total RNA was obtained from bees using TRIzol reagent for isolating biological material from organic tissue (Molecular Research Center, Inc., Cincinnati, Ohio, USA) following the manufacturer's specifications (Invitrogen, Carlsbad, Calif., USA), and RNA was synthesized into cDNA using superscript III first-strand synthesis system per manufacturer's instructions (Invitrogen, Carlsbad, Calif., USA). The DWV viral levels were then ascertained using RT-qPCR on a Bio-Rad CFX 384 Touch Real-Time PCR Detection System (Hercules, Calif., USA). As seen in FIG. 5, virus-infected bees exhibiting deformed or poorly developed wings contained about 1×10¹¹ viral load per bee; while asymptomatic bees contained about 1×10¹⁰; and healthy bees contained about 1×10⁸ viral load per bee. A one-way ANOVA with a post hoc Tukey test was used to measure significance (P<0.0001 between a & b groups). Bars represent standard error. The virus-infected bees exhibiting deformed or poorly developed wings presented with about 1000-fold increase in viral levels when compared to healthy bees. The asymptomatic virus-infected bees displayed a non-significant 100-fold difference compared to healthy bees.

The results in this example imply that the method taught in Example 1 to measure the levels of lipase activity in an insect biological sample are capable of distinguishing between terminal hives and healthy hives.

### Example 6

### Immune Responses to DWV Infection

This Example confirms that DWV injection adversely affects bee's immune response, as indicated by the fold change of Dorsal and Apidaecin in DWV-injected bees as compared to PBS-injected bees.

Honey bee pupae were injected with 10⁸ DWV viral particles in PBS, or injected with PBS alone, and total RNA isolated from individual honey bees using TRIZOL reagent for isolating biological material from organic tissue (Molecular Research Center, Inc.; Cincinnati, Ohio, USA), following the manufacturer's instructions. The concentration and purity of total RNA were determined by spectrophotometry. Differential relative expression of Dorsal and Apidaecin were tested as described by. Briefly, SYBR Green qRT-PCR was performed using primers Dorsal forward (TCGGATGGTGCTACGAGCGA; set forth in SEQ ID NO: 3); Dorsal reverse (AGCATGCTTCTCAGCTTCTGCCT; set forth in SEQ ID NO: 4); Apidaecin forward (TTTTGCCTTAGCAATTCTTGTTG; set forth in SEQ ID NO: 5), and Apidaecin reverse (GAAGGTCGAGTAGGCGGATCT; set forth in SEQ ID NO: 6).

The transcript levels of Dorsal and Apidaecin were measured 24 hours after injection, and the amount of change from non-injected was calculated using the comparative 2^{-ΔΔct} method. FIG. 6 shows that the mRNA fold change of Dorsal and Apidaecin in DWV-injected pupae was about 1; the fold change of Dorsal in PBS-injected pupae was about 2; and the fold change of Apidaecin was about 3. These results indicate that the mRNA transcript levels for both, Dorsal and Apidaecin, were significantly reduced in DWV-injected pupae when compared to PBS-injected pupae. Dorsal, a NF-κB transcription factor, was reduced approximately 2.5-fold after injection. Apidaecin, an anti-microbial peptide, typically transcribed after infection, was reduced approximately 3.5-fold after injection.

The results shown in this example correlate well with the levels of lipase activity in pupae injected with DWV or PBS measured in Example 1, and the lipid levels in healthy and deformed bees measured in Example 2.

### Example 7

### Honey Bee Worker Aging Changes in Lipase Activity

This Example demonstrates that it is possible to accurately detect a correlation between fat metabolism and bee aging when using EnzChek lipase substrate and the buffers taught in Example 1 to measure lipase activity.

Using the methods disclosed in Example 3, the lipase activity was measured in newly emerged bees, 7-day-old bees (nurses), 14-day-old bees (transitioning to forager), and 27-day-old bees (forager). The linear range and slopes of the measured lipase activities were graphed and are shown in FIG. 7. Newly emerged bees appeared to have the lowest lipase activity measured and lowest slope of the measured lipase activity when compared to the other bee stages. This is most likely due to the newly emerged bees not having consumed lipids and not needing lipase activity. The measured lipase activity and the lipase activity slope obtained for nurse bees, bees transitioning to forager, and forager bees appeared to be very similar to each other. Notably, 7 day old nurse bees had the highest lipase activity which is congruent with their caste role of feeding lipids to newly emerged bees. Day 14 bees, which are transitioning from nurse bees to forager bees had less lipase activity, and forager bees had even less lipase activity. This is developmentally important as foraging bees do not require fat burning, only carbohydrates to complete their caste role. Significant differences between the measured lipase activities in the different bee stages were determined using pair-wise Kaplan Meier linear regression (P<0.0001). Bars represent standard error.

The results in this Example indicate that the lipase activity assay taught here is accurate for nurse, middle aged, and foraging bees, while the response by newly emerged bees may be entirely different and may not be appropriate. While not wishing to be bound by theory, it is believed that bees obtain fat resources from pollen, and newly emerged bees have yet to come in contact with pollen and its fat resources.

### Example 8

### Honey Bee Worker Aging Changes in Lipid Content

This Example demonstrates that the lipid content at different stages of bee aging correlates with the fat metabolism in the bees.

The lipid levels in newly emerged bees, 7-day-old bees, 14-day-old bees, and 27-day-old bees were determined using the vanillin assay taught in Example 4. The lipid levels determined in this example were correlated with the lipase activity levels determined for the same bees in Example 7.

The lipid levels measured during honey bee aging are depicted in FIG. 8. As detected using the vanillin assay, lipid levels were lowest for newly emerged bees, and were almost twice as much for 27-day-old bees. Nurse bees (7-day-old) bees and 14-day-old bees had similar lipid levels, which were slightly higher than those measured for the 27-day-old bees. One-way ANOVA with Bonferroni's post hoc test was performed to assess significant differences (P<0.05). Bars represent standard error. These results are in agreement with the measured lipase activity shown in FIG. 7, where the lowest lipase activity measured was for newly emerged bees, and the lipase activity measured for nurse bees, bees transitioning to forager, and forager bees appeared to be very similar to each other.

The results in this Example show that measuring the lipase activity using EnzChek lipase substrate and the buffers taught herein accurately detects changes in fat metabolism with bee aging. This example provides more striking evidence that the lipase assay of the invention accurately detects fat metabolism with respect to aging.

### Example 9

### Effects of Imidacloprid on Bee Lipase Activity

This Example demonstrates that the lipase activity measured using EnzChek lipase substrate and the buffers taught in Example 4, accurately correlates to the bee's stress derived from pesticides. Honey bees challenged with sublethal doses of the neonicotinoid imidacloprid displayed significantly greater lipase activity than untreated bees, and a dose-dependent response to the pesticide.

Healthy seven and fourteen-day-old emerging adult honey bees were obtained from field colonies and were challenged with either 5 ppb or 50 ppb of the neonicotinoid imidacloprid. Same-aged bees were collected from unchallenged colonies acted as controls. The lipase activity levels were measured during a 30-minute time course, as in Example 3. A graph of the measured RFU of combined age groups was plotted against time is shown in FIG. 9. The measured lipase activity levels indicated that bees receiving sublethal 5 ppb or lethal 50 ppb imidacloprid displayed a dose-dependent response to the pesticide. While the slopes of the graphed lipase activity levels were similar for control bees and bees challenged with 5 ppb imidacloprid, the lipase activity levels of bees challenged with 50 ppb imidacloprid were significantly higher. It is likely that lethal, and not sublethal doses are detectable using the methods of the invention. Statistical significance was determined using pair-wise Kaplan Meier linear regression (P<0.01).

This example demonstrates that the lipase activity levels obtained in bees using EnzCheck lipase substrate as in Example 4, correlate with the pesticide-induced bee stress.

### Example 10

### Effects if Imidacloprid on Bee Lipid Content

This Example demonstrates the levels of lipase activity measured using EnzChek lipase substrate and the buffers taught in Example 4 correlate with the total lipid content of pesticide-treated bees, accurately predicting and detecting the bee's pesticide-derived stress.

The total lipid content on bees challenged with either 5 ppb or 50 ppb imidacloprid was measured using the vanillin assay, performed as in Example 4. The measured lipid levels (in mg/mL) were plotted, and the results are shown on FIG. 10. The total lipid content for bees treated with 5 ppb imidacloprid (P<0.01) or 50 ppb imidacloprid (P<0.05) was approximately 3-fold lower than the total lipid content measured in control bees.

The lower lipid levels determined for honey bees challenged with sublethal doses of imidacloprid correlate with the higher lipase activity levels measured using the methods of the invention. This example indicates that the methods of measuring lipase activity taught herein may be used to monitor the stress levels in pesticide-challenged bees.

We claim:

1. A kit comprising:
a fluorogenic triglyceride comprising a dye and a quencher; and
a substrate reaction buffer comprising Zwittergent detergent in Phosphate Buffered Saline (PBS).

2. The kit of claim 1, wherein the dye is a boron-dipyrromethene (BODIPY) dye; an ALEXA FLUOR dye; a PACIFIC GREEN dye; an OREGON GREEN dye; Fluorescein; fluorescein isocyanate; tetrachlorfluorescein; a CAL fluor; 4,5-dichloro-dimethoxy-fluorescein; hexachloro-fluorescein; Evans Blue; or DYLIGHT fluorescent dye.

3. The kit of claim 1, wherein the quencher is 4-((4-dimethylamino) phenyl)azo)benzoic acid (DABCYL acid); 4′-(2-Nitro-4-toluyldiazo)-2′-methoxy-5′-methyl-azobenzene-4″-(N-ethyl)-N-ethyl-2-cyanoethyl-(N,N-diisopropyl)-phosphoramidite; or a BLACK HOLE quencher.

4. The kit of claim 1, wherein the fluorogenic triglyceride comprises a BODIPY dye and a DABCYL acid quencher.

5. The kit of claim 1, wherein the substrate reaction buffer comprises about 0.005% Zwittergent detergent.

6. The kit of claim 1, further comprising:
a sample buffer comprising bovine serum albumin (BSA) and Zwittergent detergent in PBS.

7. The kit of claim 6, wherein the sample buffer comprises about 0.0015% BSA, and about 0.06% Zwittergent detergent in 4×PBS.

8. The kit of claim 6, wherein each of the sample buffer, the substrate reaction buffer, and the fluorogenic triglyceride comprising a dye and a quencher are in separate containers.

9. A method for determining lipase activity levels in an insect biological sample, the method comprising:
mixing an insect biological sample in a buffer comprising BSA, Zwittergent detergent, and PBS;
with a working solution comprising a fluorogenic triglyceride comprising a dye and a quencher and Zwittergent detergent in PBS; and
measuring the emitted fluorescence;
wherein the measured emitted fluorescence is an indication of the lipase activity in the insect biological sample.

10. The method of claim 9, wherein the dye in the fluorogenic triglyceride is a boron-dipyrromethene (BODIPY) dye; an ALEXA FLUOR dye; a PACIFIC GREEN dye; an OREGON GREEN dye; Fluorescein; fluorescein isocyanate; tetrachlorfluorescein; a CAL fluor; a DYLIGHT fluor; 4,5-dichloro-dimethoxy-fluorescein; hexachloro-fluorescein; Evans Blue; or DYLIGHT fluorescent dye.

11. The method of claim 9, wherein the quencher in the fluorogenic triglyceride is 4-((4-dimethylamino) phenyl)azo)benzoic acid (DABCYL acid); 4′-(2-Nitro-4-toluyldiazo)-2′-methoxy-5′-methyl-azobenzene-4″-(N-ethyl)-N-ethyl-2-cyanoethyl-(N,N-diisopropyl)-phosphoramidite; or a BLACK HOLE quencher.

12. The method of claim 9, wherein the fluorogenic triglyceride comprises a BODIPY dye and a DABCYL acid quencher.

13. The method of claim 9, wherein the fluorogenic triglyceride comprising a dye and a quencher is present at about 0.31 μM; about 0.62 μM; about 1.24 μM; about 2.48 μM; about 4.96 μM; about 9.92 μM; or about 19.84 μM.

14. The method of claim 13, wherein the fluorogenic triglyceride comprising a dye and a quencher is present at 2.48 μM.

15. The method of claim 9, wherein the insect biological sample is from a Coleoptera, a Lepidoptera, a Hymenoptera, or a Diptera.

16. The method of claim 15, wherein the insect biological sample is from a Hymenoptera.

17. The method of claim 16, wherein the Hymenoptera is a bee.

18. The method of claim 17, wherein the insect biological sample is from a drone bee, a worker bee, or a queen bee.

19. The method of claim 9, wherein the insect biological sample is homogenized eggs, homogenized larvae, homogenized pupae, or homogenized adult.

20. A method for determining stress in at least one test honey bee, the method comprising:
measuring the lipase activity of at least one test honey bee, and of at least one control honey bee using the method of claim 9;
comparing the measured lipase activity of the test honey bee with the measured lipase activity of the control honey bee; and
determining that the test honey bee is under stress if the measured lipase activity of the test honey bee is higher than that of the control honey bee; or the measured lipase activity in the test honey bee presents a steeper slope than the measured lipase activity in the control honey bee.

21. The method of claim 20, wherein the stress is caused by a pathogen; a pesticide; ontogeny; environmental change; or hypoxia.

22. The method of claim 20, wherein the pathogen is a deformed wing virus (DWV), Israeli acute paralysis virus (IAPV), Kashmir bee virus (KBV), acute bee paralysis virus (ABPV), black queen cell virus (BQCV), DWV, Kakugo virus, Varroa destructor virus-1/DWV-B, sacbrood virus (SBV), slow bee paralysis virus, Lake Sinai virus (LSV), Tobacco ringspot virus (TRSV), Ganda bee virus, Apis mellifera filamentous virus, Osmida cornuta nudivirus (OcNV), Bee macula-like virus, chronic bee paralysis virus (CBPV), or Scaldis River Bee Virus (SRBV).
